(19) United States
(12) Patent Application Publication
(10) Pub. No.: US 2011/0171729 A1
(43) Pub. Date: Jul. 14, 2011

(54) Method for Producing Stable Mammalian Cell Lines Producing High Levels of Recombinant Proteins

(75) Inventors: Kevin Caili Wang, Lansdale, PA (US); Shengjiang Kiu, Lafayette, CA (US)

(73) Assignee: AB MAXIS INC., Whitehouse Stations, NJ (US)

(21) Appl. No.: 12/226,938

(22) PCT Filed: May 3, 2007
(86) PCT No.: PCT/US07/10779

§ 371 Date: Feb. 22, 2011

Related U.S. Application Data
(60) Provisional application No. 60/746,490, filed on May 4, 2006.

(51) Int. Cl. C12N 5/10

(52) U.S. Cl. 435/350; 435/325; 435/358; 435/352; 435/364; 435/365; 435/369; 435/366; 435/372.3; 435/367

(57) ABSTRACT

The invention provides a novel method for generating stable mammalian cell lines with enhanced protein production capabilities, and to expression vectors and related methods for high level expression of biopharmaceutical proteins of interest.

### CROSS-REFERENCE TO RELATED APPLICATIONS

This application claims benefit under the provisions of 35 USC §119(e) of U.S. Provisional Application No. 60/746,490, filed May 4, 2006 entitled “Method to Generate Stable Cell Lines for High-level Production of Recombinant Proteins.” The disclosure of this provisional application is incorporated herein by reference in its entirety.

### TECHNICAL FIELD OF THE INVENTION

The invention generally relates to the field of recombinant protein production, more particularly to the generation of stable production cell lines for the manufacture of biopharmaceutical proteins.

### BACKGROUND OF THE INVENTION

Mammalian cells are widely used to manufacture biopharmaceutical proteins. For the production of proteins such as antibodies, which comprise complex post-translational modifications, Chinese hamster ovary (CHO) cells are typically the host cell of choice for the generation of stable mammalian production cell lines. Despite the significant advances made in recent years regarding the design and sophistication of mammalian gene expression vectors, robust production of biopharmaceutical proteins in mammalian cells is not a routine matter. Developing an expression system for large scale production of a recombinant protein requires the careful consideration of many factors, including cell growth characteristics, transgene expression levels, nature and extent of poststranslational modifications, biological activity of the protein of interest as well as regulatory issues and economic considerations.

After transfection into a mammalian host cell, an expression vector comprising a coding sequence for a biopharmaceutical protein of interest (i.e., a transgene) usually integrates randomly into the host cell's genome. Typically, in a given cell, integration occurs at a single location, as a result different cells may be expected to show integration at different positions (chromosomal locations). Due to the large size of the mammalian genome, the process of random integration generally provides transfected host cells that are characterized by variable and suboptimal expression levels. This outcome is largely attributed to the fact that there is a low probability that a transgene will randomly integrate into a genomic site that is characterized by high transcriptional activity (i.e. a hot spot). Not surprisingly, the vast majority of transfected host cells produce only low levels of the protein product encoded by the transgene. Therefore a large number of transfected host cells need to be screened in order to identify cells which are producing the protein of interest.

Amplification of the transgene is usually required to generate cell lines that are capable of producing a biopharmaceutical protein on the scale that is required for the purposes of biological evaluation and commercial production. Accordingly, expression vectors typically comprise an amplifiable marker. For example, the inclusion of the dihydrofolate reductase (DHFR) gene as an amplifiable marker in an expression vector and the use of methotrexate (MTX) for selection, can increase the gene copy number in DHFR⁻/⁻ CHO cells to 50 or more copies per cell (Omasa T et al. Journal of Bioscience and Bioengineering, Vol. 94, No. 6, pp 600-605, 2002). Well-known disadvantages of using a step-wise gene amplification strategy for the generation of production cell lines include the fact that the protein expression levels of different clones derived from an amplification protocol can cover a wide range which can exceed two orders of magnitude, the strategy requires the use of mutant cell lines, and the continued presence of MTX as a selective drug promotes cytogenetic heterogeneity which can make high copy number cell lines unstable. The latter consideration is particularly undesirable in light of the regulatory approval process for production cell lines. Moreover, the gene amplification process for high production is tedious and time consuming (possibly requiring up to four to six months). Historically, the next best alternative production system is large scale transient expression in COS cells which is quicker but more labor intensive.

Thus there exists a need in the art for methods which increase the frequency at which stable cell lines capable of high level recombinant gene expression are produced.

### SUMMARY OF THE INVENTION

The invention disclosed herein provides a method for generating stable mammalian cell lines that are capable of high-level expression of recombinant proteins by exploiting endogenous viral sequences as positions within the host cell genome as desirable targets for the integration of exogenous coding sequences. The ability to produce stable CHO cell lines that are capable of high-level production of recombinant protein provides an alternative to having to perform tedious step-wise amplification procedures or several rounds of large-scale transient COS transfections. The ability to practice the disclosed invention in CHO cells allows investigators to take advantage of the fact that CHO cells grow well in serum-free media and easily generate conditioned media on a scale which facilitates a streamlined production and purification process.

In one aspect the invention provides a method for generating a stable mammalian cell line with enhanced protein production capabilities comprising the steps of: 1) transfecting a recipient mammalian host cell harboring an integrated DNA copy of a RNA molecule within its genome with a recombinant expression vector thereby forming a transfected recipient host cell wherein the expression vector comprises: a) a DNA fragment encoding a mammalian retrovirus Gag-Pr (SEQ ID NO: 4) and b) a DNA fragment encoding a mammalian retrovirus Env fragment (SEQ ID NO: 5) positioned to flank an expression cassette comprising a DNA sequence which encodes a protein of interest; 2) isolating the transfected recipient host cell; and 3) determining the protein production capabilities of the host cell. The integrated DNA copy of an RNA molecule can be a retroviral provirus, a retrovirus-like DNA sequence, a retrotransposons, and a retrotranscript

In a particular embodiment of this aspect of the invention the host cell is a CHO ell and the DNA fragments encoding encoding mammalian retrovirus Gag-Pr fragment comprises a polynucleotide sequence consisting of (SEQ ID NO: 4) positioned 5′ to the expression cassette, and the DNA fragment encoding a mammalian retrovirus Env fragment comprises a polynucleotide sequence consisting of (SEQ ID NO: 5) positioned 3′ to the expression cassette. In alternative embodiments, the host cell can be selected from the group: Chinese hamster ovary (CHO) cells, Baby hamster kidney cells, NSO myeloma cells, monkey kidney COS cells, monkey kidney fibroblast CV-1 cells, human embryonic kidney 293 cells, human breast cancer SKBR3 cells, Human Jurket T cells, Dog kidney MDCK cells, and Human cervical cancer Hela cells.

In another embodiment, the invention provides mammalian expression vectors comprising a) a DNA fragment from CHO retrovirus gag protein gene and b) a DNA fragment from CHO retrovirus env gene positioned to flank a DNA sequence which comprises an expression cassette operably linked to regulatory sequences required to direct expression of the transgene in a mammalian host cell. The expression vectors provided by the invention are exemplified by pABMM48 (SEQ ID NO: 3) and pABME15 (SEQ ID NO: 6).

In another aspect the invention provide polynucleotide sequences encoding a CHO retroviral Gag-Pr (SEQ ID NO: 4) and a CHO retroviral Env fragment (SEQ ID NO: 5) which is capable of combining by homologous recombination with endogenous retroviral sequences harbored by CHO cells, thereby facilitating integration of the expression cassette at a site with the host cell's genome that is characterized by high transcriptional activity (i.e. a hot spot). In a particular embodiment of this aspect of the invention the polynucleotide sequence includes regulatory elements operably linked to an a expression cassette. More specifically, the invention provides expression vectors comprising DNA sequences which encode both a retroviral GagPr (SEQ ID NO: 4) and a DNA sequence encoding retroviral Env protein (SEQ ID NO: 5) positioned to flank an expression cassette encoding an antibody linked to regulatory sequences required to direct expression in a mammalian host cell.

In another aspect the invention provides mammalian host cells comprising an expression vector of the invention integrated into a site of its genome which is characterized by high transcriptional activity. As shown herein, the ability to direct (or target) an expression vector to a site within the host cell's genome that is characterized by high transcriptional activity results in the generation of recipient host cells which are characterized by enhanced protein production capability relative to production capability a host cell transfected with an expression vector devoid of the DNA fragments from mammalian retroviral sequences flanking the expression cassette.

The methods, vectors and transfected host cells disclosed and claimed herein are useful for the generation of stable mammalian (e.g., CHO cell) production cell lines cell line with enhanced recombinant protein production capabilities. Suitable production cell lines can be prepared by: 1) transfecting a recipient mammalian host cell harboring an integrated DNA copy of a RNA molecule within its genome with a recombinant expression vector thereby forming a transfected recipient host cell wherein the expression vector comprises: a) a DNA fragment encoding a mammalian retrovirus Gag-Pr and b) a DNA fragment encoding a mammalian retrovirus Env fragment positioned to flank an expression cassette comprising a DNA sequence which encodes a biopharmaceutical protein of interest, such as, but not limited to recombinant antibodies. In a particular embodiment of this aspect of the invention, the mammalian host cell is a CHO cell and the first DNA fragment encodes a CHO retrovirus Gag-Pr polynucleotide sequence consisting of the nucleotide sequence set forth in SEQ ID NO: 4 positioned 5′ to an expression cassette, and the second DNA fragment encodes a CHO retrovirus Env fragment polynucleotide sequence consisting of the nucleotide sequence set forth in SEQ ID NO: 5 positioned 3′ to the expression cassette.

The invention also provides a method for producing high levels of recombinant proteins in stable mammalian production cell lines comprising the steps of: 1) transfecting a recipient mammalian host cell harboring an integrated DNA copy of a RNA molecule within its genome with a recombinant expression vector thereby forming a transfected recipient host cell wherein the expression vector comprises: ) a DNA fragment encoding a mammalian retrovirus Gag-Pr (SEQ ID NO: 4) and b) a DNA fragment encoding a mammalian retrovirus Env fragment (SEQ ID NO: 5) positioned to flank an expression cassette comprising a DNA sequence which encodes a recombinant protein; and 2) isolating the transfected recipient host cell; and 3) culturing the isolated host cell of step 2) under conditions suitable for enhanced protein production.

In a particular embodiment, the invention provides a method for producing high levels of an antibody. Without needs of gene amplification steps, stable cell lines generated using this invention have the capability to produce antibody at the productivity level of more than 10 pg/cell/day, preferably 20 pg/cell/day, more preferably 30 pg/cell/day. At those level of productivity, it will produce grams/L of antibody with optimized cell culture condition

The invention further provides a method for modulating the efficiency of mammalian cell transfection comprising transfecting a recipient mammalian cell harboring an endogenous retroviral sequence in its genome with an expression vector comprising an expression cassette operably linked to a polynulcleotide sequence consisting of at least one recombinant polynucleotide sequence capable of combining with the endogenous retroviral sequence by homologous recombination. In a particular embodiment of this aspect of the invention the method provides a means of increasing the frequency of transfected recipient host cells capable of producing high levels of a biopharmaceutical protein of interest.

### BRIEF DESCRIPTION OF THE DRAWINGS

FIG. 1 is a schematic representation of the mammalian expression vector pABMM1 (SEQ ID NO: 1). It was derived from pcDNA6/His-5A, and comprise an ampicillin-resistant gene (AMPr) for antibiotic selection in E. coli, a plasmid replication on (pUC ori), a expression cassette of DHFR and neomycin genes for selection in mammalian cells (pSV40-DHFR-IRES-Neomycin-polyA), antibody light chain expression cassette (pCMV-light chain-polyA) and antibody heavy chain expression cassette (pCMV-heavy chain-polyA).

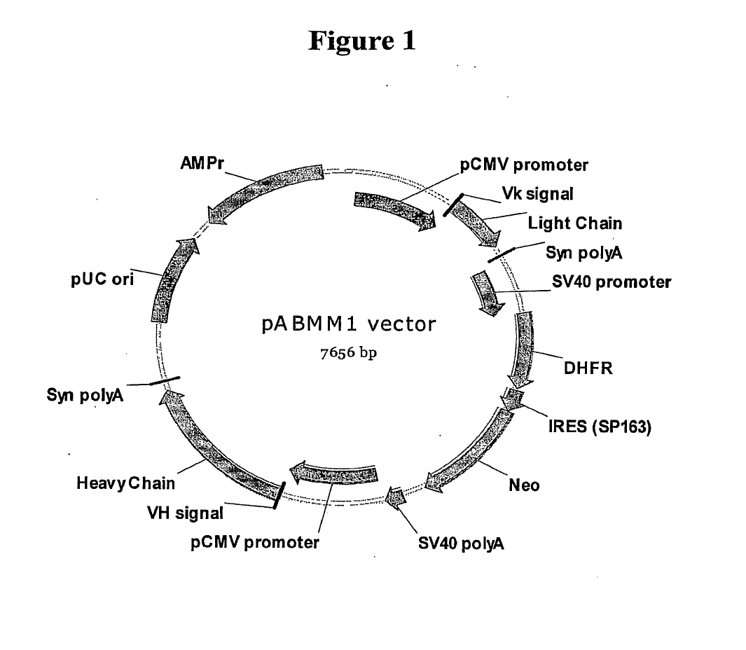

FIG. 2 is a schematic representation of the mammalian expression vector pABMM72 (SEQ ID NO: 2). It was derived from pABMM1 vector, and comprise an ampicillin-resistant gene (AMPr) for antibiotic selection in E. coli, a plasmid replication on (pUC ori), an expression cassette of antibody heavy chain (pCMV-heavy chain-polyA), and an expression cassette for antibody light chain, with the attachments of DHFR and neomycin genes by two IRES sequences (pCMV-light chain-SP163-DHFR—SP163-Neomycin-polyA). The VH and Vk genes were cloned into this vector for expression of an antibody against VEGF.

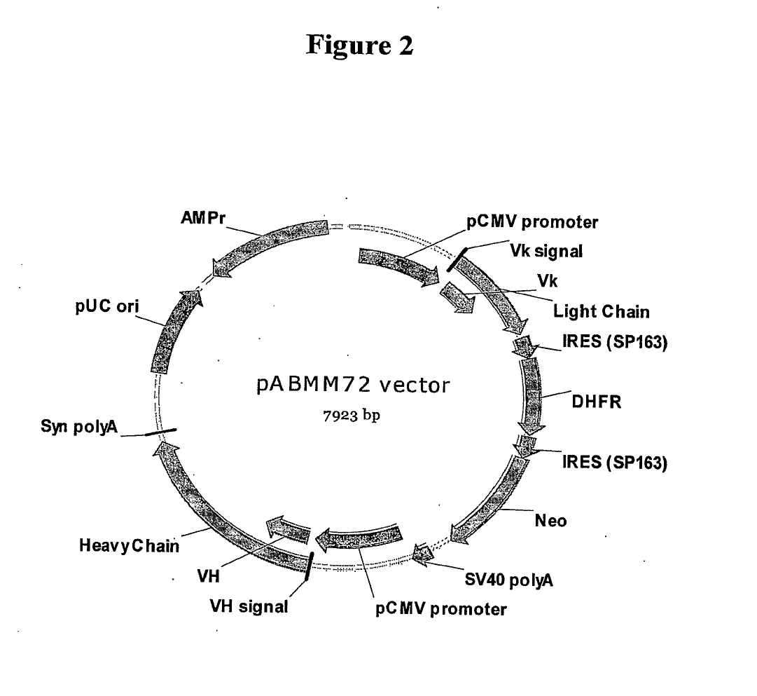

FIG. 3 is a schematic representation of the mammalian expression vector pABMM48 (SEQ ID NO: 4). It was derived from pABMM1 vector, and comprise an ampicillin-resistant gene (AMPr) for antibiotic selection in E. coli, a plasmid replication on (pUC ori), a expression cassette of DHFR and neomycin genes for selection in mammalian cells (pSV40-DHFR-IRES-Neomycin-polyA), antibody light chain expression cassette (pCMV-light chain-polyA) and antibody heavy chain expression cassette (pCMV-heavy chain-polyA). Moreover, a DNA fragment from CHO retrovirus Gag-Pr gene was inserted upstream of light chain expression cassette, and a DNA fragment from CHO retrovirus Evn gene was located downstream of antibody heavy chain expression cassette. The VH and Vk genes were cloned into this vector for expression of an antibody against VEGF.

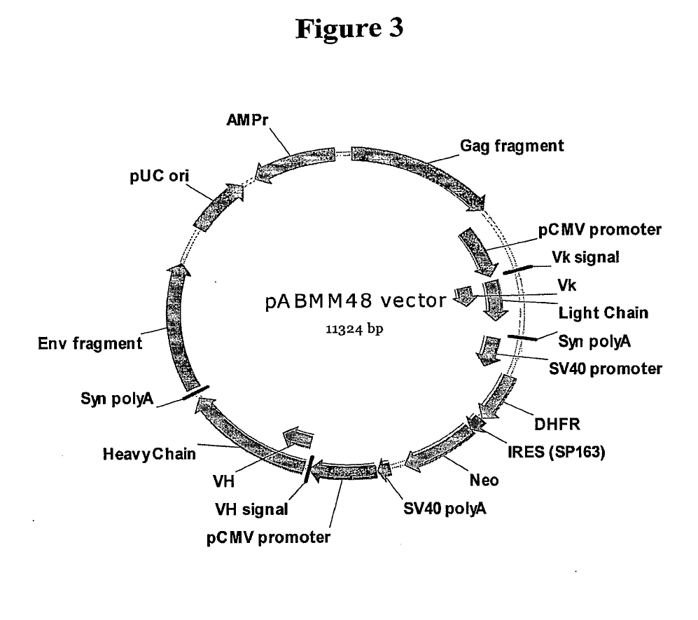

FIG. 4 is a schematic representation of the mammalian expression vector pABME15 (SEQ ID NO: 7). It was derived from pABMM48 vector originally, and comprise an ampicillin-resistant gene (AMPr) for antibiotic selection in E. coli, a plasmid replication on (pUC ori), an expression cassette for antibody light chain, with the attachments of neomycin genes by an IRES sequence (pCMV-light chain-SP163-Neomycin-polyA), and an expression cassette of antibody heavy chain, with attachment of DHFR gene by an IRES sequences (pCMV-heavy chain-Sp163-DHFR-polyA). Moreover, a DNA fragment from CHO retrovirus Gag-Pr gene was located upstream of light chain expression cassette, and a DNA fragment from CHO retrovirus Env gene was located downstream of antibody heavy chain expression cassette. The VH and Vk genes were cloned into this vector for expression of an antibody against VEGF.

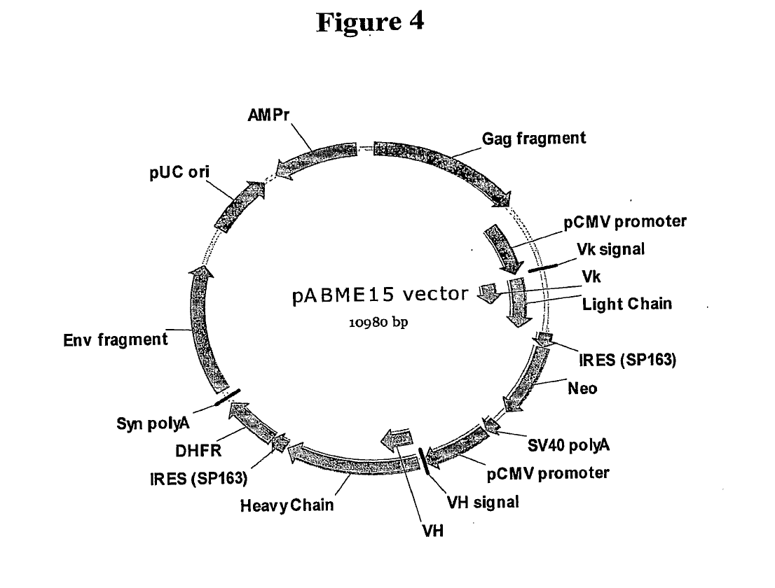

FIGS. 5A and 5B provide graphic representations of the results of anti-human antibody ELISA assays for the detection of stable cell lines in 96-well plates. FIG. 5A illustrates different levels of recombinant antibody expression in culture supernatants obtained from CHO cell lines generated from vector pABMM72. FIG. 5B shows expression level of recombinant antibody in the culture supernatant generated from vector pABMM48.

FIG. 6 shows the antibody productivity of different cell lines generated from the expression vectors with (M48 clones) or without (M72 clones) retroviral sequences.

FIG. 7 shows the result of antibody production from one cell line generated with a pABMM48 vector comprising retroviral sequences over the course of 3 days (i.e., 72 hrs of culture).

### DETAILED DESCRIPTION OF THE INVENTION

As used in this specification and claims, the singular form “a,” “an,” and “the” include plural references unless the context clearly dictates otherwise.

As used herein the term “production cell line,” refers to host cells which have been transformed or transfected with expression vectors constructing using recombinant DNA techniques and which contain sequences encoding recombinant proteins. Transformation refers to modifying a recipient host cell by the addition of a nucleic acid, such as an expression vector. Transfection refers to the introduction of a nucleic acid into a recipient host cell by chemical means or electroporation. Expressed proteins will preferably be secreted into the culture supernatant, depending upon the design of the expression vector (e.g., inclusion of a secretory leader).

As used herein, the term “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as gene product. If the polynucleotide is derived from genomic DNA, expression may include splicing of the mRNA in a eukaryotic cell.

Homologous recombination is a type of genetic rearrangement that occurs through the breakage and rejoining of DNA molecules within a stretch (some hundreds to thousands of base pairs) of identical or very similar (i.e., homologous) sequences. Homologous recombination of a DNA vector into a region of genome can be done in almost any cell type but occurs at a low frequency. Enhancement of the frequency of homologous recombination can be achieved by (1) linearization of the vector DNA; (2) maximization of the sequence homology to recombination; (3) modification of the 3′ hydroxyls of the transfected DNA with dideoxynucleotides.

Polynulcleotides or nucleic acids of the invention may be in the form of RNA or in the form of DNA, which DNA includes cDNA, genomic DNA or synthetic DNA. The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A “vector” is a nucleic acid molecule, preferably self-replicating, which transfers an inserted nucleic acid molecule into and/or between host cells. An “expression vector” is a polynucleotide sequence which, when introduced into an appropriate host cell, can be transcribed and translated into a polypeptide(s). An “expression system” usually connotes a suitable host cell comprised of an expression vector that can function to yield a desired expression product.

The terms “gene,” “gene fragment” and “coding sequence” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof.

As used herein the term“heterologous” means derived from a genotypically distinct entity from the rest of the entity to which it is being compared. For example, a promoter removed from its native coding sequence and operatively linked to a coding sequence other than the native sequence is a heterologous promoter. The term “heterologous” as applied to a polynucleotide, a polypeptide, means that the polynucleotide or polypeptide is derived from a genotypically distinct entity from that of the rest of the entity to which it is being compared. For instance, a heterologous polynucleotide or antigen may be derived from a different species origin, different cell type, and the same type of cell of distinct individuals.

The term “recombinant” as applied to a polynucleotide means that the polynucleotide is the product of various combinations of cloning, restriction and/or ligation steps, and other procedures that result in a construct that is distinct from a polynucleotide found in nature.

As used herein the terms “operably linked” or “operatively linked” are used to refer the DNA sequences which are juxtaposed in a manner such that the components so described are in a relationship permitting them to function in their intended manner. For example, a promoter is operably linked to a coding sequence if it controls the transcription of the sequence; or a ribosome binding site is operably linked to a coding sequence if it is positioned so as to permit translation. DNA for a signal sequence (secretory leader) is operably linked to DNA for a polypeptide if it is expressed as a precursor which participates in the secretion of the polypeptide. Generally, operably linked means contiguous

As used herein the terms “polypeptide”, “peptide” and “protein” are used interchangeably herein to refer to polymers of amino acids of any length. The polymer may be linear, cyclic, or branched, it may comprise modified amino acids, and it may be interrupted by non amino acids. The terms also encompass amino acid polymers that have been modified; for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs.

As used herein the term “antibody” refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, chimeric antibodies, humanized antibodies and fragments thereof. Non-limiting examples of antibody fragments include a Fab fragment consisting of the VL, VH, CL and CH1 domains; (4) an Fd fragment consisting of the VH and CH1 domains; (5) an Fv fragment consisting of the VL and VH domains of a single arm of an antibody; (6) an F(ab′)2 fragment, a bivalent fragment comprising two Fab fragments linked by a disulfide bridge at the hinge region, (7) a diabody consisting of two identical single chain Fv with shorter linker; (8) a ccFv antibody consisting of Fv stabilized by a pair of coiled-coil domains interaction.

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues.

The site of transgene integration is known to have a significant effect on the transcription rate of the recombinant gene (referred to in the art as the position effect). Several strategies have been used to overcome the negative effects of random integration and gene silencing. For example, some investigators have reported success overcoming the unwanted consequences associated with random integration by flanking transgenes with protective cis-regulatory elements such as insulators, boundary elements, or scaffold/matrix attachment regions (Bode J et al, Crit. Rev Eukaryot Gene Expr. 6(2-3):115-38. 1996). These elements are included in expression vectors in an attempt to provide an artificial genomic environment that is believed to favor high transcriptional activity. Therefore, these elements will overcome the position effect in cell's genome, and make the expression of transgene relatively position-insensitive. These approaches have reported variable success at increasing the frequency of transformants capable of high-level expression of recombinant genes (Phi-Van L et al, Mol Cell Biol, 10(5):2302-7, 1990; Scippel A E et al, U.S. Pat. No. 5,731,178, Mar. 24, 1998; Girod P A et al, Biotechnol Bioeng. 5; 91(1):1-11, 2005; Mermod N et al, U.S. Pat. No. 7,129,062, Oct. 31, 2006).

An alternative strategy to avoid position effects is to design and implement a gene targeting strategy for the purpose of targeting transgene integration to transcriptionally active regions of the host cell's genome. The transcriptionally active regions are normally identified from the cells cable of high-level expression of recombinant proteins. These high-level producing cell lines are isolated from screening large mount of transfected cells. One of the examples is to use of a DNA vector (Neospla) containing a translationally impaired dominant selectable marker for selection of cell lines cable of high-level expression of recombinatant proteins (U.S. Pat. No. 5,648,267). In the Neospla vector, the selection marker neomycin phosphotransferase (Neo) gene has been artificially split into two exons. Furthermore, U.S. Pat. No. 5,830,698 described the use of two vectors (maker plasmid, and target plasmid) containing three neo exons for site specific integration by homologous recombination. The function of maker plasmid containing one neo exon is to create a target sites in host cells. After homologous recombination with targeting vector containing two neo exonx, the high-level producing cells with functional neo gene will be selected out from other low-level cells. Following this approach, cell lines with antibody productivity of 0.3-4.5 pg/cell/day were isolated.

The invention disclosed herein is based on targeting a preexisting endogenous DNA sequences within the host cell genome that is known to represent a possible hot spot and therefore is predicted to be likely to direct high-level expression of an exogneous coding sequence as a target for transgene integration by homologous recombination. The introduction of a transgene comprising a coding sequence flanked by nucleotide sequences designed to be homologous to endogenous hot spot sequences promotes the integration of the exogenous coding sequence by homologous recombination. This strategy provides a novel method to exploit endogenous retroviral sequences present in a mammalian host cell for the production of stable cell lines characterized by a high-level expression of the transgene and resulting production of a biopharmaceutical protein of interest.

Endogenous retrovirus and retrovirus-like sequences are present in almost all mammal genomes. Recent findings from sequencing human genome reveal that around 8-10% of the human and mouse genome appears to consist of sequences with similarity to infectious retrovirues, which contain at least three genes, including gag (encoding structural proteins), pol (viral enzymes), and env (surface envelope proteins), as well as long terminal repeats (LTRs). (Griffiths D J, Genome Biology, 2:1017.1-1017.5, 2001; Nature 420: 520-562, 2002). Accumulated data suggests that retroviral integration is not totally random in the genome. In the case of HIV infection, current studies indicated that retroviral integration favors active genes. Analysis of all reported integration sites showed that 92.5% integration sites are flanked with matrix-attached regions (MARS) (Biochem Biophys Res Commun. 2004, 322:672-7). Bode J et al (Biochemistry, 1996, 35: 2239-52) have observed that the retroviruses selectively target a scaffold- or matrix attached regions (S/MARs).

Retroviruses are characterized by a high degree of overall structural similarity (5′-LTR-gag-pol-env-LTR reading frames) an ability to reverse transcribe their RNA genome into DNA which integrates into host chromosomes and acts as a stable genetic element. Endogenous retroviruses are known to exist in numerous species, for example, published studies indicate that Chinese Hamster Ovary (CHO) cells contain transcriptionally active full-length type C proviral sequences (Lie Y S, et al, J. Virol. 68(12):7840-9. 1994). It has been estimated that CHO cells harbor between approximately 100-300 copies per cell of type C retrovirus sequences (Dinowitz et al, Dev Biol Stand. 76:201-7. 1992). It is unlikely to have multiple integrations due to low frequency of recombination ( 1/100- 1/1000).

In contrast to numerous rodent cell lines, CHO cells do not product detectable levels of infectious retrovirus. However type-C retrovirus like particles have been detected in CHO cells by electron microscopy which have been attributed on an endogenous origin as opposed to retroviral infection. Although the underlying mechanism is not entirely understood, it is apparent that retroviral proviruses have a tendency to selectively integrate into preferred chromosomal sites. In particular, it has been observed that the process of retroviral integration favors transcriptionally active genes.

The invention disclosed in this application exploits these retroviral sequences as hot spots for targeted (or directed) integration of an expression vector. More specifically, it utilizes preexisting or endogenous viral sequences as the targets for the production of stable mammalian production cell lines. For example, CHO retrovirus sites can be targeted for directed integration of a transgene comprising an expression cassette which codes for a biopharmaceutical protein of interest. As shown herein, flanking antibody genes with gag and Env genes fragments that are capable of homologous recombination with endogenous CHO retroviral sequences, results in the production of stable cell lines expressed more then 10-fold higher IgG1 proteins compared to the level of antibody that is produced from host cells that are transfected with a vector which did not comprise retroviral targeting sequences.

Results of the transfection experiments presented in Examples 8-10 of the Detailed Description which utilize expression vectors that comprise DNA sequences designed to direct homologous recombination with endogenous retroviral provirus sequences establish that the expression vectors of the invention produce more transfectants (recipient transfected host cells) that are capable of high level antibody production than transfectants produced with vectors comprising all of the same regulatory elements and expression cassette in the absence of the retroviral DNA sequences.

Examples of polypeptides that can be expressed in the mammalian expression system of the invention can include any biopharmaceutical protein of interest, including but not limited to antibodies (including humanized antibodies or fragments thereof), human cytokines, growth factor, growth factor receptor, enzymes, such as Interleukin-2, Interferon, Human Isulin, human growth hormone, Erythropoietin, GM-CSF, G-CSF, Follitropin alpha, tissue plasminogen activator, Platelet cells derived growth factor, Tumor necrosis factor, TNF receptor, glucocerebrosidase, alpha-galactosidase; and recombinant vaccines such as hepatitis-B antigen, diphteria toxin protein.

Polypeptides of interest can be produced by any means through use of the methods disclosed herein including transformation or transfection of mammalian host cells with a vector construct disclosed herein. Host cells of the present invention may be propagated or cultured by any method known or contemplated in the art, including but not limited to growth in culture tubes, flasks, roller bottles, shake flasks or fermentors. Isolation and/or purification of the polypeptide products can be conducted in accordance with the method described in Example 7 of this disclosure or by any means known in the art.

While not wishing to be bound by a particular theory, it is believed that the higher frequency of high-producer cell lines is attributed to the nature and location of the integration sites utilized by the expression vectors. More specifically, it is believed that the expression vectors of the invention direct the transgenes to transcriptional hot spots. This effect is considered to be a consequence of the process of retroviral integration, which is known to favor actively transcribed genes. The ability to design expression vectors that are capable of selectively introducing an expression cassette into a transcriptional hot spot of a mammalian host cell's genome will facilitate the generation of stable production cell lines and the development of manufacturing processes suitable for the production of biopharmaceutical proteins.

Elements of the expression vectors designed for use to generate a production cell line and the transformation protocol selected to introduce the expression vector into suitable host cells will depend on the nature of the mammalian cell culture system that is being used to manufacture the protein of interest. Those of skill in the art are aware of numerous different protocols and host cells, and can select an appropriate system for production of a desired protein, based on the requirements of their chosen cell culture system. Current data suggested the extracellular retrovirus-like particles of CHO cells are products of endogenous provirus elements present in the Chinese hamster germline (Anderson K P et al, Dev. Biol. Stand. 75:123-132, 1991). Most endogenous retroviruses are highly transcribed during early zygotic divisions and in germ cells, resulting in more copies of proviral integrations. In order to against harmful consequences of endogenous retrovirus, numerous cellular defense strategies has involved to counteract virus amplication, which include DNA methylation of promoters to block transcription, and DNA or RNA editing to alter coding sequences. As matter of factor, the retroviral gag and env fragments amplified from CHO genomic DNA for this invention have stop codons inside the coding regions. Therefore, the endogenous viral promoter in the viral 5′ long terminal repeat (LTR) may not be an active promoter for transgen expression when it integrate downstream of LTR sequence. The suitale translational regulatory elements such as enhancer, promoter, sequence encoding suitable mRNA ribosomal binding sites are required to operably linked to the transgene.

To achieve homologous recombination with endogenous retrovirus genome, two pieces of sequences with homology to viral sequences are needed to flank the transgene. The reported minimum homology for efficient recombination in mammalian cells is 200 bp. It is generally belief that longer length of homology, higher efficiency could be. Preferably, 1 kb to 5 kb of DNA homologous fragments could be used for the recombination. Most retroviruses shear the gene structure: 5′LTR-gag-pol-env-3′LTR, within the size of 8 to 11 kb. In principle, any two fragments from two ends of viral genome with the length of few hundreds to few thousands by can be used for homologous recombination, except the whole viral genome, which will generate full viral particles harmful for downstream purification. The flaking viral sequences can be fully synthetic, or be amplified from host genome as did in this invention.

The optimal expression of eukaryotic cDNA sequence requires a careful consideration of several structural features, including the 5′ and 3′ untranslated sequences flanking the expression cassette and the nucleotide context around the translation inititation codon. In eukaryotic cells translation of most mRNAs is initiated according to a “scanning model.” However, the scanning model of translation initiation does not apply to many viral and some cellular messages which are translated in a cap-independent manner at internal sites known as internal ribosomal entry sites (IRES). It is believed that cellular trans-acting proteins bind to the IRES element and facilitate ribosome biding and translation initiation. Robust polycistronic vectors, such as the vectors of the invention, typically utilize IRES elements to facilitate internal ribosome binding to the second and subsequent transcription unit.

The invention also provides homologous recombination vectors that are capable of directing the integration of exogenous coding sequences (i.e., genes) into hot spots within the genome of a suitable host cell thereby leading to the generation of a production cell line which is capable of high levels of protein production making it suitable for use in the manufacture of a biopharmaceutical protein.

Generally speaking recombinant expression vectors include cDNA-derived or synthetic polynucleotide (e.g., DNA) sequences encoding a protein sequence, operably linked to suitale translational regulatory elements derived from mammalian and/or viral genes. Such regulatory elements typically include a transcriptional promoter, a sequence encoding suitable mRNA ribosomal binding sites, and sequences which control the termination of transcription and translation. Mammalian expression vectors may also comprise nontranscribed elements such as origins of replication, a suitable promoter and enhancer linked to the gene (e.g., coding sequence) to be expressed, other 5′ or 3′ flanking sequences such as a polyadenylation site, splice donor and acceptor sites and transcriptional termination sequences. Optionally, an origin of replication that confers the ability to replicate in a host and a selectable gene to facilitate recognition of transformations may also be included.

It is well known that transcriptional and translational control sequences in expression vectors designed for use in transforming mammalian cells may be obtained from viral sources. For example, commonly used promoters and enhancers are derived from Simian Virus 40 (SV40), human cytomegalovirus, Polyoma or Adenovirus 2. Viral genomic promoters, control and/or signal sequences can be utilized to drive expression, provided such control sequences are compatible with the host cell. DNA sequences derived from the SV40 viral genome, for example, SV40 origin, early and late promoter, enhancer, splice and polyadenylation sites may be used to provide other genetic elements required for expression of an exogenous (i.e. heterologous) DNA sequence.

Conventional retroviral vector must comprise a number of cis-acting viral elements, which typically include (1) a promoter in the viral 5′ long terminal repeat (LTR); (2) a viral packaging signal (ψ or E) to direct incorporation of vector RNA into virions; (3) signals required for reverse transcription, including a transfer RNA-binding site (PBS) and polypurine tract (PPT) for initiation of first- and second-strand DNA synthesis, and a long terminal repeated (LTR) region at both ends of the viral RNA required for transfer of DNA synthesis between templates; and (4) short, partially inverted repeats located at the termini of the viral LTRs required for integration. In contrast the expression vectors of the invention may comprise some of the above viral elements in the ends of transgene, but not all of the elements listed above, and on this basis are by definition not viral vectors. Moreover, the expression vectors RNA in this invention will not be packaged into viral particles for cell infections.

Various mammalian cell culture systems can be employed to produce a production host cell line, due to existing of endogenous retroviruses in almost all mammal cells. Examples of suitable host cells include cell lines harboring endogenous retroviruses sequences, retrovirus-like DNA sequences, retrotransposons, and retrotranscripts, such as Chinese hamster ovary (CHO) cells, Baby hamster kidney cells, NSO myeloma cells, monkey kidney COS cells, monkey kidney fibroblast CV-1 cells, human embryonic kidney 293 cells, human breast cancer SKBR3 cells, Human Jurket T cells, Dog kidney MDCK cells, Human cervical cancer Hela cells. Due to the variation of retroviruses in the cell lines from different species, the retrovirus sequences for expression vector may be different. Preferably, the sequences flanking transgenes are amplified from the individual cell line for transfection, or synthezed from the viral sequence isolated from same cell line

The examples provided herein establish that the Chinese Hamster Ovary (CHO) cell line CHO-DG44 is suitable for use in the methods of the invention. DHFR⁻ CHO cells which are auxotrophic for glycine, thymidine and hypoxanthine are commonly used host cells, and can be transformed to a DHFR⁺ phenotype using DHFR cDNA as an amplifiable dominant marker. In this invention, DHFR selectable marker was built in the expression vectors, and can be used for gene amplification if need. General speaking, other CHO cell lines, such as CHO-k1, CHO-S, GS-CHO, with same genomic background are also suitable for recombinant protein expression using the vectors described in examples of this invention.

The term transfection refers to a variety of art-recognized protocols for the introducing foreign DNA into host cellst (see Kaufman, R. J. Meth. Enzymology 185:537 (1988)). Selection of a transformation protocol will depend upon the host cell and the nature of the transgene and protein product. The basic requirements of a suitable protocol are first to introduce the exogenous DNA encoding the protein of interest into the host cell, and the ability to isolate and select host cells that have incorporated the heterologous DNA in a stable, expressible manner.

Commonly used method for introducing exogenous DNA into host cells inucle calcium phosphate precipitation or DEAE-dextran-medicated transfection. Alternatively, electroporation can be used to introduce DNA directed into the cytoplasm of a host cell, or a reagent (e.g., Lipofectin® Reagent or Lipofectamine® Reagent, Gibco BRL, Gaithersburg, Md.) capable of forming lipid-nucleic acid complexes or liposomes which facilitates uptake of nucleic acid into host cells when the complex is applied to cultured cells can be used.

A method of amplifying the gene of interest is also desirable and typically involves the use of a selection gene which confers a selectable phenotype. Generally speaking a “selection gene” is a gene that confers a phenotype on cells that express the gene as a detectable protein. A Commonly used example of selection genes include but are not limited to, antibiotic resistance genes. For example, useful dominant selecteable markers include microbially derived antibiotic resistance genes, which confer resistance to neomycin, kanamycin or hygromycin when the drug (or selection agent) is added exogenously to the cell culture.

The process of gene amplification is routinely used to increase copy number of the transgene comprising the expression cassette which encodes the biopharmaceutical protein of interest. For example, if the dihydroforate reductase (DHFR) gene amplification system in (DFR⁻) Chinese Hamster Ovary (CHO) cell line is used, transfected CHO cells are cultivated in a medium which contains the toxic drug (i.e. MTX). This drug inhibits the enzyme which is expressed by the encoded marker gene. Selection is based on the fact that most of the cells will not survive in the presence of MTX, but a few cells, in particular the cells containing higher number of transfected selectable marker genes will survive. The copy number of the objective genes is correlated with, and increases with the number of marker genes. Therefore, the process of increasing the MTX concentration, simultaneously selects for high-productive cell lines.

The step-wise amplification is extremely important to low-level producing cells with number of pg/cell/day below 1, which are the typical cells generated from regular methods. In order to achieve high-level productivity, the number of transgene in cells need be amplified to 30 to 50 copies with high concentration of MTX (i.e. 1 mM) by multiple steps, dependent on their original productivity. Even the relatively high-level producing cells generated from neospla vector with around 3 pg/cells/day productivity need be amplified to round 10 copies transgene per cell with low concentration of MTX (50 nM), to produce therapeutic antibody (U.S. Pat. No. 5,830,698). The expression vectors of this invention have two selectable markers: neomycin and MFR. With MTX in culture medium, the transgene can be amplified if need. However, the step-wise amplification process may not need in most case, due to the original high-level productivity of cells isolated from the vector with retrovirus flanking sequences. For example, the cell line IDI isolated in Example 10 has ˜20 pg/cell/day productivity of antibody in a non-optimized condition (regular shaker flask). It is possible to achieve much higher pg/cell/day number (i.e 30 to 40 pg/cell/day) in a fermentor with optimized culture condition, that is higher enough for production of therapeutic recombinant proteins. A comparison of the cell line productivity using this the methods of this invention relative to the productivity reported using the methods reported in the US patents indicated in the first column is provided in Table 1.

TABLE 1

| | | antibody productivity |
|---|---|---|
| Methods | gene amplification | (pg/cell/day) |
| U.S. Pat. No. 5,648,267 | No | 2.5-3.6 |
| U.S. Pat. No. 5,830,698 | No | 0.3-4.5 |
| U.S. Pat. No. 5,830,698 | Yes | 10-18 |
| This invention | No | 3.4-20 |

The examples and figures provided with this disclosure illustrate results obtained using the compositions and methods of the present invention to generate stable mammalian cell lines. The following examples are meant to be illustrative of an embodiment of the present invention and should not limit the scope of the invention in any way. A number of modifications and variations will be apparent to the skilled artisan from reading this disclosure. Such modifications and variations constitute part of the invention.

The practice of the present invention employs, unless otherwise indicated, conventional techniques of cell biology, molecular biology, cell culture and the like which are in the skill of one in the art. All publications and patent applications cited in the specification are indicative of the level of skill of those skilled in the art to which this invention pertains and are hereby incorporated by reference in their entirety.

### Example 1

### pABMM1 Vector Construction for IgG Expression in Mammalian Cells

pABMM1 (SEQ ID NO; 1) was created from a backbone vector pcDNA6/His-5A by following three steps. First, the DNA fragment coding an expression cassette for two selection makers DHFR/neomycin was assembled through overlapping PCR from 5 DNA fragments, which are synthetic polyA, SV40 promoter amplified by PCR from vector pcDNA3, DHFR cDNA amplified by RT-PCR from the RNA of CHO-K cell, synthetic SP163 (Internal Ribosome Entry site from 5′ UTR of VEGF), and Neomycin cDNA amplified by PCR from vector pcDNA3. The DNA fragment of PolyA-SV40 promoter-DHFR-SP163-Neomycin was cloned into vector pcDNA6/His-5A by NheI and Drain restriction sites.

Second, a DNA fragment coding for human antibody signal peptide, partial Jk segment and human k constant region was inserted downstream of pCMV promoter in the modified pcDNA 6 vector described above. This DNA fragment was generated from, PCR assembly, in which the signal sequence was synthesized from human antibody VK VI-A14 with NheI site by silent mutation, Ck constant regain was amplified by RT-PCR from the human spleen mRNA (Clonetech).

The third step was to insert an antibody heavy chain expression cassette downstream of DHFR/Neomycin selection markers by SalI site. This heavy chain fragment, including a CMV promoter, a heavy chain signal sequence, a partial JH segment, and CH1 to CH3 of human IgG1, was generated through PCR assembly. The heavy chain signal sequence was synthesized from human antibody VH3_{—}3-23 (DP47), with the AflII site sequence coding for amino acids LK. The cDNA for human IgG 1 antibody CH1 to CH3 was amplified by RT-PCR from human spleen mRNA.

### Example 2

### pABMM72 Vector Construction for IgG Expression in Mammalian Cells

paBMM72 (SEQ ID NO: 2) was derived from pABMM1. The SV40 promoter for DHFR/Neomycin in pABMM1 vector was replaced by an internal ribosome entry sequence SP163 in BamHI/NcoI sites, resulted in one transcription for the antibody light chain, DHFR, and neomycin driven from single CMV promoter in pABMM72 vector. Furthermore, an anti-VEGF antibody heavy and light chain variable region genes VH and Vk were cloned into this vector respectively by NheI/BsiWI and AflII/XhoI sites.

### Example 3

### pABMM48 Vector Construction for IgG Expression in Mammalian Cells

pABMM48 (SEQ ID NO: 3) was constructed from pABMM1 vector as described below. First, the antibody (anti-VEGF antibody) heavy and light chain variable region genes VH and Vk were cloned into this vector respectively by NheI/BsiWI and AflII/XhoI sites. Second, a 1540 bp of DNA fragment for retrovirus Gag-Pr gene fragment was inserted into BglII site upstream of CMV promoter of light chain. The retrovirus Gag-Pr DNA fragment (SEQ ID NO: 4) was amplified from CHO-DG44 cells by PCR. It has two open reading frames coding truncated gag-pr proteins (amino acid 30-381, and 383-545, with a stop codon between), and 61% ( 317/518) identity with murine leukemia virus gag-pol polyprotein (full length of 1736 amino acids). Third, a 1462 bp of DNA fragment (SEQ ID NO: 5) for retrovirus Env gene was amplified from CHO-DG44 cells by PCR, and was inserted into SalI site downstream of heavy chain expression cassette. The Env fragment has two ORFs coding two truncated envelope proteins (amino acid 72-305, and 339-492), and 58% ( 118/202) identity with murine leukemia virus gPr80 envelope protein (full length of 652 amino acids).

### Example 4

### pABME15 Vector Construction for IgG Expression in Mammalian Cells

The pABME15 vector was derived from pABMM79 vector (SEQ ID NO: 6). Vector pABMM79 was created from pABMM48 by removing one Bell site at 1561 bp and one SalI site at 7628, and inserting one NotI site at 3029 bp and one AscI site at 7500 bp. This step is to introduce unique restriction site for each functional segment in the expression vector. In order to attach neomycin expression with antibody light chain cassette, the XbaI-XhoI fragment from pABMM79 was cloned into pBluescript SK(+) vector first, then the NotI-MluI fragment from this modified pBluescript SK(+) vector was used to replace the corresponding fragment in pABMM79. This step generated an expression cassette of pCMV-L chain-SP163-neomycin-SV40 polyA in new vector. Furthermore, a DNA fragment for SP163-DHFR was inserted downstream of antibody heavy chain by AscI sites. This step created cassette of pCMV-heavy chain-SP163-DHFR-synthetic PolyA in pABME15 vector (SEQ ID NO: 7). The attachments of selection marker neomycine to antibody light chain expression, DHFR to antibody heavy chain expression is to assure the expression of both heavy chain and light chain, and to prevent the possibility of loss of any chain of antibody from DNA rearrangements.

### Example 5

### Culture and Transfection of CHO-DG44

The Chinese Hamster Ovary cell line CHO-DG44 was cultured in suspension with serum-free medium, which contains 90% CHO-S-SFM II (Invitrogen/GIBCO) and 10% CHO Ex-cell serum free medium (JRH), with 8 mM Glutamine and 0.3% Penicillin/Streptomycin (Invitrogen/GIBCO). Briefly, cells were seeded to a 250 ml shaker flask containing 50 ml serum-free growth medium, grew at shaking speed of 130 rpm in a 37° C., 5% CO2 incubator. Cells were fed with fresh medium very day, and split into 2 shaker flask every another day. Only the cells at their mid log phase with >95% cell viability and 30%˜50% dividing cells were used for transfection.

The transfection of cells with vector DNA was done by electroporation. First, the vector DNA was linearized with restriction enzyme BglII (for pABMM72) or HindIII (pABMM48 and pABME15). The cells was washed twice with ice-cold PBS by spinning 5 min at 1000 rpm, and re-suspended in ice cold PBS at density of 1×10⁷ cells/ml. 400 ul of cells were then mixed with 20 ug of vector DNA, and transfer to an ice-cold 0.4 cm cuvette for electroporation. The Biorad Gene Pulser II was set at capacity of 500 V Max, voltage of 0.350 KV, and CAP of 600 uF. After the shock, the cells were put at room temperature for 15 min, and transferred to 50 ml centrifuge tube with 30 ml culture medium, then plated into three 96-well-plates for growth and selection.

### Example 6

### ELISA Assay for Antibody Detection

Anti-IgG ELISA assay was used for quantification of human IgG in CHO cell cultures. Briefly, the 96-well plate was coated with antibody against human IgG Fc (Calbiochem) at 1 ug/ml in 0.1 M carbonate buffer, pH9.6, and incubated for overnight at 4° C. The plate was then blocked with 5% milk in PBST for 1 hr at room temperature. The cell culture supernatants with serial dilution or were added to each well for 1 hr incubation at room temperature. In the meanwhile, human IgG proteins (Sigma) with known concentrations were added to the same plate in parallel as standards. After three times wash with PBST, the 2^{nd} Antibody (goat anti-human IgG kappa chain—HRP conjugate, Sigma) in 3% milk-PBST was added for one hour incubation at room temperature. Finally, ABTS substrate in stable peroxide substrate buffer (Pierce) was added for color development. The absorbance at 405 nm was measured after 30 min of development on a′ plate reader (SpectraMax Plus, Molecular Devices). The linear standard curve of IgG was generated within the range 0-12.5 ng/ml. The concentrations of human IgG in cell culture supernatants were calculated from the standard curve according to its OD readouts.

### Example 7

### Antibody Production and Purification

The stable clones selected for IgG production were grown in production medium that contains CHO Ex-cell medium (JRH), 8 mM Glutamine (GIBCO), 0.3% antibiotic (GIBCO). Briefly, cells were seeded at density of 0.2-0.3 million cells/ml in 250 ml shaker flask with 30 ml production medium. The shaker was set at speed of 130 rpm in a 37° C., 5% CO2 incubator. Cells were fed very day with fresh medium. The culture medium was changed very 2 days, until culture reached accumulatively to 1 liter. The cells were then transferred to a 3 liter shaker flask with 1 liter of production medium at cell density of 1 million/ml, and grew at shaking speed of 80 rpm, in a 37° C., 5% CO2 incubator. The cells were fed with 50 ml fresh production medium every 1-2 days until cell density reach at 3-4 million/ml. The temperature for culture was then shifted to 33° C. to promote IgG production. Cells were still fed with 50 ml fresh production medium every one or two days, until cell viability lower than 80-85%. The supernatants were harvested, and cells were removed by centrifugation.

The supernatants harvested from cell cultures were first filtered through 0.4 um filter, and concentrated to 100-200 ml through Pall tangential flow filtration device using a 50 kDa cut-off filter. The sample was then loaded to a MabSelect (protein A) column (25 mm×200 mm, CV=98.2 mL, Pmax=40) at a flow rate of 5 ml/min. After wash with 5 column volumes of Buffer A (50 mM HEPES, 150 mM NaCl, pH 7.0) and 5 column volumes of Buffer B (50 mM Sodium acetate, pH 5.0), IgG protein was eluted with 5 column volumes of Buffer C (100 mM Acetic acid, 22 mM Phosphoric acid, pH 2.0). The fractions of human IgG at OD280 pick were collected and combined, and the pH was neutralized with 5% fraction volume of 1 M Tris-HCl buffer, pH 9. The precipitate formed during pH neutralization was removed by 30 min centrifugation at 10,000 rpm. Purified antibodies were further analyzed using size-exclusion high performance liquid chromatography (SE-HPLC) on an Agilent 1100 HPLC system (Agilent) equipped with TSK-GEL SuperSW3000 column (Tosoh Bioscience). Briefly, 10 ul of diluted sample was loaded to a TSK-GEL Super SW3000 column at flow rate 0.25 ml/min. The phosphate buffered saline PBS with 0.05% (w/v) sodium azide was used for mobile phage. The 280 nm UV signal was monitored to determine protein picks. Molecular weight marker proteins (29 kD-660 kD) for gel filtration (Sigma) were used as standards in the assay.

### Example 8

### Analysis of Vector Integration

Genomic DNAs are extracted from the stable cell line. Briefly, cells are harvested by 10 min spin at 1200 rpm, and washed twice with PBS, once with nuclei lysis buffer (10 mM Tris EDTA, pH 8.0, 0.4M NaCl). After re-suspend in 3 ml of nuclei lysis buffer, cells are lysized by adding 100 μl Proteinase K (10 mg/ml) and 400 μl of 10% SDS, and incubated overnight at 45° C. The supernatant from the lysate is then used for DNA preparation. The genomic DNAs are precipitated by adding 1/10 the total volume 3 M sodium acetate (pH 5.2) and 3 times total volume cold 100% isopropanol, and washed with 70% ethanol. The dry pellet of DNA is resuspend in 200 ul H2O.

The genomic DNA is digested with the restriction enzyme EcoRI, and ligated into the EcoRI site of a pre-digested Lambda DASH II vector, which is part of the Lambda DASH II/EcoRI Vector Kit (Stratagene, USA). Packaging extracts are used to package the recombinant lambda phage following the instruction of the manufacturer (Gigapack III Gold Packaging Extract; Stratagene, USA). Of the resulting library, about 1×10⁶ plaque forming units (pfu) are plated onto NZY agar plates, using XL1-Blue MRF′ bacteria strain as a phage host and incubated overnight at 37° C. The library is amplified to prepare a large, stable quantity of a high-titer stock of the library following the instruction of the manufacturer.

The library is plated out at 50 000 pfu/plate on large 150 mm NZY agar plates and incubated overnight at 37° C. A nitrocellulose membrane (Stratagene, USA) is placed onto each NZY agar plate for 2 minutes to transfer the phage particles to the membrane. A needle is used to prick through the membrane and agar for orientation. The membrane is denatured in a solution of 1.5 M NaCl and 0.5 M NaOH for 2 min, which is followed by neutralization for 5 min in 1.5 M NaCl and 0.5 M Tris-HCl, pH 8. The membrane is rinsed for 30 sec in a solution containing 0.2 M Tris-HCl (pH 7.5) and 2×SSC solution buffer. The DNA is finally cross-linked to the membrane using an UV transilluminator. The genomic DNA library is screened by Southern blot analysis. Two DNA probes containing human antibody heavy and light chain constant region are labeled, and used for screening. The positive clones isolated from screening are analyzed by DNA sequencing to determine the sequences of integration site.

### Example 9

### Stable Cell Line Generation with pABMM72 Vector

CHO-DG44 cells were grown in serum-free medium with HT in a 250 ml shaker flask at speed of 130 rpm in a 37° C., 5% CO2 incubator. The cells reached the mid log phase with 96% cell viability and 40% dividing cells after 5 days culture. After wash twice with ice-cold PBS, the CHO-DG44 cells were re-suspended in ice-cold PBS at density of 1×10⁷ cell/ml, and incubated for 15 min on ice. 400 ul of cells were then mixed with 20 ug of pABM72 vector DNA linearized at BglII site, and transferred to an ice-cold cuvette for electroporation.

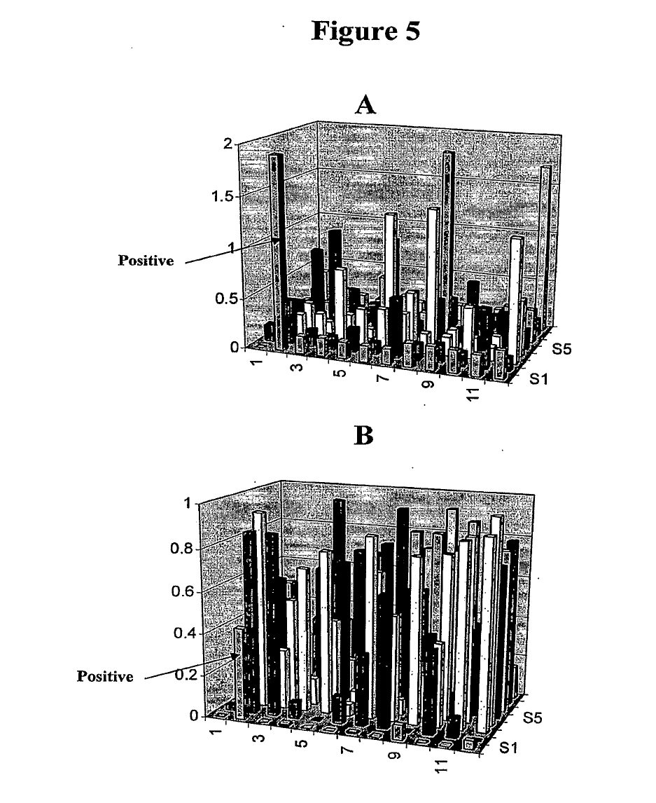

The electroporations were carried out using a Biorad Gene Pulser II with the setting of the capacity of 500 V Max, voltage of 0.350 KV, and CAP of 600 uF. A total four electroporations were performed. After the shock, the cells were put at room temperature for 15 min, and transferred to 50 ml centrifuge tube with 30 ml culture medium. The transfected cells were washed with growth medium, and plated into three 96-well plates for each electroporation. Two days (48 hrs) after of electroporation, the cells were selected by adding growth medium with HT and 0.5 mg/ml G418. The culture medium was changed by 50% every 3-4 days. The growth clones were visible under microscope after 2 to 3 weeks of selection. 100 ul of culture supernatants from each well were taken for expression screening (typical data from one 96 well plate was showed in FIG. 5A). No gene amplification process was carried out in those experiments.

The anti-human Fc/anti-human K chain ELISA as described in example 5 was performed to evaluate the expression level of individual clones. The clones with high ELISA readings were then transferred to 24-well plates, 6-well plates, and T75 flasks for growth. A total 50 clones were picked up for this cell amplification process. Supernatants harvested from 48 hr cultures in T75 flasks were used in a quantitative ELISA. Cell line productivity was evaluated by calculating a pg/cell/day human IgG production level for each clone. Table 2 provides the results obtained from the top 6 clones.

TABLE 2

| Clone | pg/cell/day | Estimate production mg/L* |
|---|---|---|
| 13-3-D6 | 1.09 | 54.50 |
| 10-3-C5 | 0.67 | 33.50 |
| 4-1-C9 | 0.43 | 21.50 |
| 13-3-F9 | 0.35 | 17.50 |
| 10-2-E11 | 0.28 | 14.00 |
| 14-1-G10 | 0.15 | 7.50 |

*10 culture at cell density of 5 million/ml.

The highest clone 13-3-D6 produced human IgG at level of 1.09 pg/cell/day, which can provides a total production of 54 mg IgG per liter after 10 days of shaker flask culture at cell density of 5×10⁶ cells/ml.

### Example 10

### Stable Cell Line Generation with pABMM148 Vector

CHO-DG44 cells were cultured in suspension with serum-free medium, containing 0.90% CHO-S-SFM II (Invitrogen/GIBCO) and 10% CHO Ex-cell serum free medium (JRH), with HT. Briefly, cells were seeded to a 250 ml shaker flask containing 50 ml serum-free growth medium, with shaking at speed of 130 rpm in a 37° C., 5% CO2 incubator. Cells were fed with fresh medium very day, and split into 2 shaking flask every another day. The cells at their mid log phase with 96% cell viability and 38% dividing cells were used for transfection. After wash twice with ice-cold PBS, the CHO-DG44 cells were re-suspended in ice-cold PBS at density of 1×10⁷ cell/ml, and incubated for 15 min on ice. 400 ul of cells were then mixed with 20 ug of pABM48 DNA linearized at HindIII site, and transferred to an ice-cold cuvette for electroporation.

The electroporations were carried out by Biorad Gene Pulser II with the setting of the capacity of 500 V Max, voltage of 0.350 KV, and CAP of 600 uF. A total four electroporations were done. After the shock, the cells were put at room temperature for 15 min, and transferred to 50 centrifuge tube with 30 ml culture medium. The transfected cells were washed with growth medium, and plated into three 96-well-plates for each electroporation. Two days (48 hrs) after electroporation, the cells were selected by growth medium with HT and 0.5 mg/ml G418. The culture medium was changed every 3-4 days. The growth clones were visible under microscope after 2-3 weeks of selection. 100 ul of culture supernatants from each well were taken for expression screening. Totally, around 1000 clones were screened. But no gene amplification process was carried out in those experiments.

The anti-human Fc/anti-human K chain ELISA as described in example 5 was performed to evaluate the expression level of individual clones. The ELISA data in FIG. 5B establishes that transfection with pABMM48 results in a higher frequency of high producer clones. The clones with high ELISA readings were then transferred to 24-well plates, 6-well plates, and T75 flasks for growth. A Total 50 clones were selected for this cell amplification.

The supernatants of 48 hr cultures of T75 flasks were used for quantitative ELISA to determine a pg/cell/day (pcd) production level of human IgG by dividing the antibody concentration with the cell number at 48 hr and 2 days. The pcd number might be underestimated, due to use only the cell number at 48 hr, which is the highest number during two days culture. The top 9 clones with high-expression of human IgG1 were showed in the table 3.

TABLE 3

| Clones | pg/cell/day | Estimate production mg/L* |
|---|---|---|
| 1D1 | 12.2-20** | 610-1000 |
| 2B4 | 4.6 | 230 |
| 2C1 | 10 | 500 |
| 10-2-E10 | 3.8 | 190 |
| 2D2 | 4.43 | 221.5 |
| 9-3-G3 | 3.4 | 170 |

*10 culture at cell density of 5 million/ml.
**measured from shaker flask

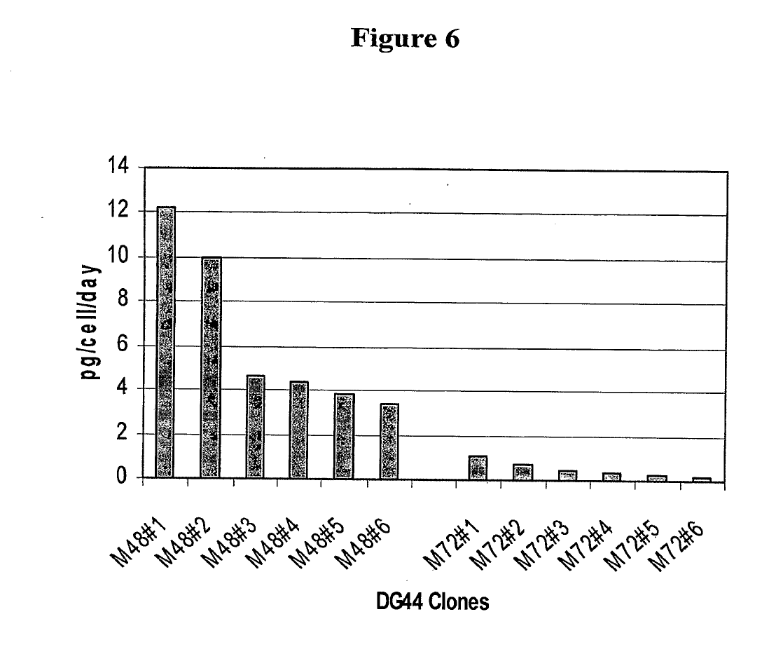

The highest clone 1D1 produced human IgG at level of 12.2 pg/cell/day (measured from T75 flask), which can result in the production of 610 mg IgG after 10 days of shaker flask culture at cell density of 5×10⁶ cells/ml. A comparison of expression level from the stable cell lines generated from pABMM72 (which does not include retroviral sequences) reveals that clones produced from the use of the pABMM48 vector (which comprises retroviral seqs) are characterized by more than 10-fold higher IgG productivity (FIG. 6).

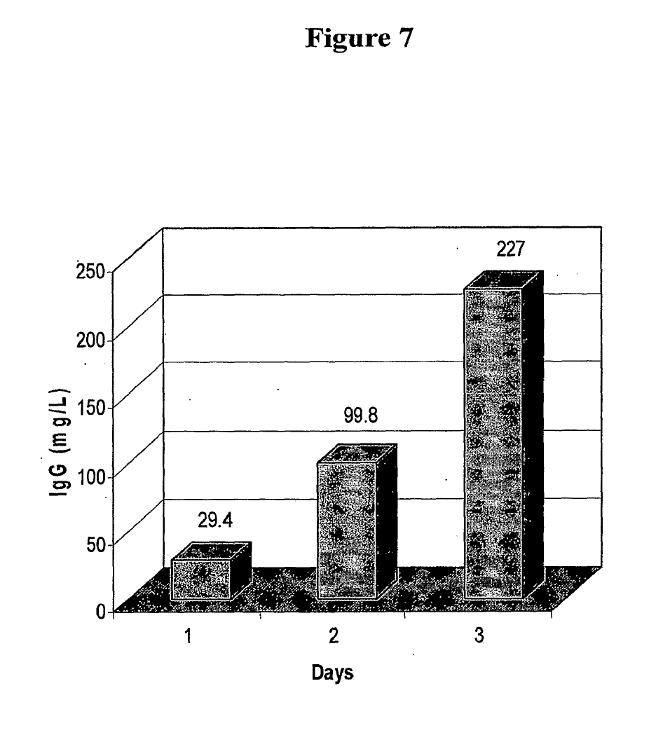

The best clone, 1D1, was further scaled up for antibody production in a shaker flask. Briefly, cells were seeded at density of 0.3 million cells/ml in 250 ml shaker flask with 30 ml production medium. The shaker was set at speed of 130 rpm in a 37° C., 5% CO2 incubator. Cells were fed very day with 10% fresh medium. The medium was changed very 2 days, until culture reached to 0.5 liter. Then the cells were transferred into a 3 liter shaker flask with 0.4 liter of production medium at cell density of 1 million/ml, and grew at shaking speed of 80 rpm, in a 37° C., 5% CO2 incubator, with feeding of 50 ml fresh production medium every days until cell density reach at 4 million/ml. The temperature for culture was then shifted to 33° C. for IgG production. Cells were fed with 50 ml fresh production medium every day. After 3 days, 0.55 liter of supernatants were harvested. The supernatants from day 1, day 2, day 3 were collected for quantitative ELSA. The data in FIG. 7 showed the production of IgG for day 1 to day 3 is 29.4 mg/L, 99.8 mg/L and 227 mg/L.

The IgG protein in the culture supernatant was purified as described below. The supernatants were first filtered through 0.4 um filter, and concentrated to 100 ml through Pall tangential flow filtration device using a 50 kDa cut-off filter. The sample was then loaded to a MabSelect (protein A) column (25 mm×200 mm, CV=98.2 mL, Pmax=40) at a flow rate of 5 ml/min. After wash with 5 column volumes of Buffer A (50 mM HEPES, 150 mM NaCl, pH 7.0) and 5 column volumes of Buffer B (50 mM Sodium acetate, pH 5.0), IgG protein was eluted with 5 column volumes of Buffer C (100 mM Acetic acid, 22 mM Phosphoric acid, pH 2.0). The fractions of human IgG at OD280 pick were collected and combined, and the pH was neutralized with 5% fraction volume of 1 M Tris-HCl buffer, pH 9. The precipitate formed during pH neutralization was removed by 30 min centrifugation at 10,000 rpm.

The final purified human IgG from this 0.55 liter of 3 days culture was 163 mg, which was calculated to give 20 pg/cell/day productivity, and transformed to 1 g of IgG production from 10 days of shaker flask culture at cell density of 5×10⁶ cells/ml. Furthermore, 10 ul of diluted purified IgG protein was loaded to a size exclusion column TSK-GEL Super SW3000 (Tosoh) for analytical HPLC assay. HPLC Data confirmed the purity with only signal IgG pike.

These results establish that the CHO production cell lines transfected with the pABMM48 vector (comprising retroviral sequences) produced more antibody than clones produced from vector without the retroviral sequences. The data presented herein shows that the use of expression vectors comprising the endogenous retrovirus sequences of the present invention, compared to conventional expression vectors, increases the cell productivity of recombinant antibody from <1 pg/cell/day obtained using a regular vector to 20 pg/cell/day. This represents a 20 fold increase in cell productivity.

### REFERENCES

1. Omasa T et al. Gene amplification and its application in cell and tissue engineering (review), Journal of Bioscience and Bioengineering, Vol. 94, No. 6, pp 600-605, 2002
2. David J Griffiths Endogenous retroviruses in the human genome sequence Genome Biology (review), 2:1017.1-1017.5, 2001
3. Smithies O et al, Insertion of DNA sequences into the human chromosomal b-globin locus by homologous recombination, Nature 317, 230-234, 1985
4. Koduri R K et al, An efficient homologous recombination vector pTV(I) contains a hot spot for increased recombinant protein expression in Chinese hamster ovary cells, Gene. 280(1-2):87-95, 2001
5. Koduri R K et al, Expression vectors containing hot spot for increased recombinant protein expression in transfected cells, U.S. Pat. No. 6,800,457, Oct. 5, 2004
6. Johnson C N et al, Matrix attachment regions as targets for retroviral integration. Virol J. 19; 2:68, 2005
7. Kulkarni A et al, HIV-1 integration sites are flanked by potential MARs that alone can act as promoter, Biochem Biophys Res Commun. 322 (2):672-7, 2004,
8. Mielke C et al, Anatomy of Highly Expressing Chromosomal Sites Targeted by Retroviral Vectors, Biochemistry, 35: 2239-52, 1996
9. Bode J et al, Scaffold/matrix-attached regions: topological switches with multiple regulatory functions. Crit. Rev Eukaryot Gene Expr. 6(2-3):115-38. 1996
10. Phi-Van L et al, The chicken lysozyme 5′ matrix attachment region increases transcription from a heterologous promoter in heterologous cells and dampens position effects on the expression of transfected genes. Mol Cell Biol, 10(5):2302-7, 1990
11. Scippel A E et al, Attachment-elements for stimulation of eukaryotic expression systems, U.S. Pat. No. 5,731,178, Mar. 24, 1998.
12. Girod P A et al, Use of the chicken lysozyme 5′ matrix attachment region to generate high'producer CHO cell lines. Biotechnol Bioeng. 5; 91(1):1-11, 2005
13. Mermod N et al, Matrix attachment regions and methods for use thereof, U.S. Pat. No. 7,129,062, Oct. 31, 2006.
14. Reid G G et al, Comparison of electron microscopic techniques for enumeration of endogenous retrovirus in mouse and Chinese hamster cell lines used for production of biologics. J Virol Methods. 108(1):91-6. 2003
15. Lie Y S, et al, Chinese hamster ovary cells contain transcriptionally active full-length type C proviruses. J Virol. 68(12):7840-9. 1994
16. Dinowitz et al, Recent studies on retrovirus-like particles in Chinese hamster ovary cells. Dev Biol Stand. 76:201-7. 1992
17. Anderson K P et al, Defective endogenous retrovirus-like sequences and particles of Chinese hamster ovary cells. Dev Biol Stand. 75:123-32. 1991
18. International Mouse Genome Sequencing Consortium (2002) Initial sequencing and comparative analysis of the mouse genome. Nature 420: 520-562
19. Mitchell E R, Impaired dominant selectable marker sequence and intronic insertion strategies for enhancement of expression of gene product and expression vector systems comprising same. U.S. Pat. No. 5,648,267, Jun. 15, 1997
20. Mitchell E R et al, Method for integrating genes at specific sites in mammalian cells via homologous recombination and vectors for accomplishing the same, U.S. Pat. No. 5,830,698, Nov. 3, 1998.

1. A method for generating a stable mammalian cell line with enhanced protein production capabilities comprising the steps of:
1) transfecting a recipient mammalian host cell harboring an integrated DNA copy of a RNA molecule within its genome with a recombinant expression vector thereby forming a transfected recipient host cell wherein the expression vector comprises: a) a DNA fragment encoding a mammalian retrovirus Gag-Pr fragment and b) a DNA fragment encoding a mammalian retrovirus Env fragment positioned to flank an expression cassette comprising a DNA sequence which encodes a protein of interest; 2) isolating the transfected recipient host cell and 3) determining the production capability of the cell line.

2. The method of claim 1 wherein the host cell is a CHO cell and the DNA fragment encoding encoding a mammalian retrovirus Gag-Pr fragment comprises a polynucleotide sequence consisting of (SEQ ID NO: 4) positioned 5′ to the expression cassette.

3. The method of claim 1 wherein the host cell is a CHO cell and the DNA fragment encoding a mammalian retrovirus Env fragment comprises a polynucleotide sequence consisting of (SEQ ID NO: 5) positioned 3′ to the expression cassette.

4. The method of claim 1 wherein the host cell is selected from the group: Chinese hamster ovary (CHO) cells, Baby hamster kidney cells, NSO myeloma cells, monkey kidney COS cells, monkey kidney fibroblast CV-1 cells, human embryonic kidney 293 cells, human breast cancer SKBR3 cells, Human Jurket T cells, Dog kidney MDCK cells, and Human cervical cancer Hela cells.

5. The method of claim 1 wherein the DNA copy of an RNA molecule is selected from a retroviral provirus, a retrovirus-like DNA sequence, a retrotransposons, and a retrotranscript.

6. (canceled)

7. (canceled)

8. (canceled)

9. (canceled)

10. (canceled)

11. (canceled)

12. A host cell comprising an expression vector comprising a) a DNA fragment from a mammalian retrovirus Gag-Pr and b) a DNA fragment from a mammalian retrovirus Env gene positioned to flank an expression cassette comprising a DNA sequence which encodes an expression cassette which comprises a protein of interest operably linked to regulatory sequences required to direct expression in a mammalian host cell.

13. The host cell of claim 12 wherein the host cell is a CHO cell.

14. A host cell according to claim 12 wherein the cell is characterized by enhanced protein production capability relative to production capability a host cell transfected with an expression vector devoid of the DNA fragments from CHO retrovirus Gag-Pr and CHO retrovirus Env gene flanking the expression cassette.

15. A method for generating a stable mammalian cell line with enhanced antibody production capabilities comprising the steps of: 1) transfecting a recipient mammalian host cell harboring an integrated DNA copy of a RNA molecule within its genome with a recombinant expression vector thereby forming a transfected recipient host cell wherein the expression vector comprises: a) a DNA fragment encoding a mammalian retrovirus Gag-Pr fragment and b) a DNA fragment encoding a mammalian retrovirus Env fragment positioned to flank an expression cassette comprising a DNA sequence which encodes an antibody; 2) isolating the transfected recipient host cell and 3) determining the antibody production capability of the cell line.

16. The method of claim 15 wherein the host cell is a CHO cell and the DNA fragment encoding a mammalian retrovirus Gag-Pr comprises (SEQ ID NO: 4) and the DNA fragment encoding a mammalian retrovirus Env fragment comprises (SEQ ID NO: 5).

17. (canceled)

18. (canceled)

19. A method for modulating the efficiency of mammalian cell transfection comprising transfecting a recipient mammalian cell harboring an endogenous retroviral sequence in its genome with an expression vector comprising an expression cassette operably linked to a polynulcleotide sequence consisting of at least one recombinant polynucleotide sequence capable of combining with the endogenous retroviral sequence by homologous recombination.

20. The method of claim 19 wherein the mammalian cell is a CHO cell and the recombinant polynucleotide sequences capable of combining with the endogenous retroviral sequence by homologous recombination comprise polynucleotide sequences encoding a CHO retroviral Gag-Pr (SEQ ID NO: 4) and a CHO retroviral Env fragment (SEQ ID NO: 5) operably linked to an expression cassette.
